(19) United States
(12) Patent Application Publication
(10) Pub. No.: US 2005/0152934 A1
(43) Pub. Date: Jul. 14, 2005

(54) Methods for removing deposits of impurities from the scalp and hair

(75) Inventor: Alessandro Casero, Como (IT)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: FARMAKA s.r.l.

(21) Appl. No.: 11/077,449

(22) Filed: Mar. 10, 2005

Related U.S. Application Data
(62) Division of application No. 10/315,815, filed on Dec. 9, 2002.
Application No. 10/315,815 is a continuation of application No. PCT/IT01/00289, filed on Jun. 6, 2001.

(30) Foreign Application Priority Data
Jun. 9, 2000 (IT) MI2000A 001300

(51) Int. Cl. A61K 7/00

(52) U.S. Cl. 424/401

(57) ABSTRACT

Cosmetic compositions are provided for removing deposits of impurities on scalp and hair. The compositions contain some emollient substances, a granular substance adapted to mechanically remove the deposits, and a chelating agent having the task of facilitating disaggregation of the deposits. The compositions can be used to prevent the formation of the deposits and is therefore an important means for maintaining the fair hygiene of hair and scalp, an essential condition for their good health.

### CROSS-REFERENCE TO RELATED APPLICATIONS

This application is a division of U.S. patent application Ser. No. 10/315,815, filed Dec. 9, 2002, now abandoned, which in turn is a continuation of International Application No. PCT/IT01/00289, filed Jun. 6, 2001, which was published in the English language on Dec. 13, 2001, under International Publication No. WO 01/93817 A1 and the disclosure of which is incorporated herein by reference.

### BACKGROUND OF THE INVENTION

The present invention relates to cosmetic compositions for the care of hair, and in particular to a composition allowing removal of deposits of impurities often arising on scalp and hair.

It is known that deposits of impurities consisting of variously originating substance, such as furfuraceous scales, degradation products of sebum and sweat, dust and various inorganic substances, such as calcium, deriving from water used in normal cleaning, may grow on hair and scalp. In particular the last named inorganic item favors the aggregation of all these substances, thus making the resulting deposits particularly hard and sticky to hair and scalp. In addition, because of the presence of calcium, the deposits are insoluble in water and cannot be disaggregated and cleared away by means of usual detergents.

The production of such deposits results immediately in a worsening of the aesthetic appearance of hair that shortly becomes rough and opaque. A more serious consequence resides in the deposits clogging the pores and hair follicles, thus preventing cutaneous respiration and causing weakening and, in the long run, loss of hair.

### BRIEF SUMMARY OF THE INVENTION

It is therefore an object of the present invention to provide a product able to remove deposits of impurities from hair and scalp, thus eliminating the causes of their weakening. This object is attained by means of a cosmetic composition, preferably in the form of a creamy emulsion, whose principal features are emollient substances, an abrasive granular substance, a calcium chelating agent, a keratolytic compound, and an antibacterial agent.

A first advantage of the cosmetic compositions according to the present invention resides in that, due to the calcium chelating agent and granular substance therein, they can be used in eliminating one of the reasons of hair loss and therefore in fighting baldness. According to a particular embodiment, a composition in conformity with the present invention also comprises a compound having a keratolytic effect and able to favor the disaggregating action carried out on noxious deposits.

Another advantage of cosmetic compositions according to the present invention resides in that, by aiding the separation and removal of dead cells, they also have a marked anti-dandruff action.

A further advantage of cosmetic compositions according to the present invention resides in that they also contain some emollient substances which contribute to improve the aesthetic appearance of hair by making it bulky, strong and glossy, and assist in hair combing.

A particular advantage of compositions according to the present invention resides in that, due to the antibacterial compounds contained therein, they prevent the degradation of sebum and sweat and avoid the formation of deposits of these.

Further advantages and features of the cosmetic compositions according to the present invention will appear to those skilled in the art from the following detailed description of an embodiment thereof.

### DETAILED DESCRIPTION OF THE INVENTION

The cosmetic compositions according to the present invention comprise a creamy emulsion of conventional type, further comprising a granular substance and a chelating agent.

Such a granular substance is adapted to allow for removal of noxious deposits formed on hair and scalp by applying a delicate massage on it through the fingers. Granular substances usable in the compositions according to the present invention are all the substances conventionally used in products for the cleaning of skin, for example microcrystalline silica, calcium carbonate, tin oxide, magnesium stearate, synthetic resins, microcrystalline cellulose, Gransilk™ and the like.

The chelating agent has the task of complexing the calcium ions by separating them from the noxious deposits, so as to further aid in their disaggregation. Various chelating agents known in the art may be included in the composition according to the present invention, such as ethylenediaminetetraacetic acid, sodium heptagluconate, or etidronic acid. Preferably, ethylenediaminetetraacetic acid or its salt is used in a preferred amount of 0.01% to 01% by weight of the composition according to the present invention.

According to a particular embodiment, the composition in conformity with the present invention also comprises a compound having a keratolytic action that affects and dissolves the epidermis particles enclosed in the noxious deposits, thus making the disaggregating action more effective on the latter. Such a keratolytic compound is preferably salicylic acid, which is present in compositions according to the present invention in amount of 0.001% and 0.05% by weight.

According to a particular embodiment, compositions in conformity with this invention also comprise an antibacterial compound having the task of preventing decomposition of sebum and sweat, so as to oppose the formation of noxious deposits. The antibacterial compound is preferably chlorexidine, which is present in the composition in an amount of 0.01% to 1% by weight.

Due to this last ingredient, the composition according to this embodiment of the invention is able to prevent the formation of noxious deposits on scalp and hair, which formation, as stated above, is linked to the decomposing bacterial activity on some organic residues. The composition can therefore also be used to preserve the proper bacterial flora of the scalp, which is essential to maintain the good health of hair as well.

A non limiting example of a preparation according to the invention is as follows.

### EXAMPLE

In a founding furnace the following ingredients are heated to 65° C. until a clear mixture is obtained:

| Ingredient | Amount (kg) |
|---|---|
| Cetylstearyl alcohol | 56 |
| Cetyl alcohol | 30 |
| Cetylstearyl ether polyethoxylate | 20 |
| Paraffin oil | 20 |
| Lanolin | 10 |
| Laurylmethicone | 20 |
| Methylparaben | 2.0. |

In an emulgator are then heated to 70° C.:

| Ingredient | Amount (kg) |
|---|---|
| Demineralized water | q.s. to 1000 |
| EDTA | 2 |
| Sodium heptagluconate | 2 |
| Etidronic acid | 1.5 |
| Salicylic acid | 0.2 |

Both phases thus obtained are then homogenized for 15-20 minutes, and to the resulting emulsion there are subsequently added:

| Ingredient | Amount (kg) |
|---|---|
| Gransilk ™ | 3 |
| Maize starch | 20 |
| Microcrystalline cellulose | 20 |

A de-aeration and homogenization step is then effected until a homogeneous mass is obtained. The mass is afterwards allowed to cool down to 40° C., and under swift agitation are added:

| Ingredient | Amount (kg) |
|---|---|
| Cetyltrimethylammonium chloride | 30 |
| Chlorexidine bichlorhydrate | 1 |
| Perfume | q.s. |

A gentle agitation under vacuum is continued, and cooling down to ambient temperature is carried out by setting the pH at 4-4.5 with possible regulation by means of lactic or citric acid.

The cosmetic composition thus obtained is a creamy emulsion to be used as a “mask” by gently massaging it on wet hair before or after a shampoo, allowing it to act for 3-10 minutes and then rinsing with water.

Although a particular embodiment of the present invention has been described, possible changes and/or additions may be introduced by those skilled in the art, yet remaining in the scope of the invention.

1. A method for removing deposits of impurities from scalp and hair, the method comprising applying to the scalp or hair a composition comprising an emollient substance, an abrasive granular substance, a calcium chelating agent, a keratolytic compound and an antibacterial agent.

2. The method according to claim 1, wherein the granular substance is selected from the group consisting of microcrystalline silica, calcium carbonate, zinc oxide, magnesium stearate, microcrystalline cellulose and synthetic resins; the calcium chelating agent is selected from the group consisting of sodium heptagluconate, etidronic acid, and ethylenediaminotetraacetic acid; the keratolytic compound is salicylic acid; and the antibacterial agent is chlorexidine.

3. The method according to claim 2, wherein the calcium chelating agent is ethylenediaminotetraacetic acid present in an amount of 0.01% to 0.1%, the salicylic acid is present in an amount of 0.001% to 0.05%, and the chlorexidine is present in an amount of 0.01% to 1%, all amounts being by weight of the total composition.

4. The method according to claim 1, wherein the composition is in a form of a creamy emulsion used as a mask.

5. The method according to claim 1, wherein the composition is applied to the hair by a gentle massage.

6. The method according to claim 5, wherein the composition is applied to wet hair.

7. The method according to claim 6, wherein the composition is allowed to act for 3 to 10 minutes before rinsing with water.
